(19) United States
(12) Patent Application Publication
(10) Pub. No.: US 2022/0356138 A1
(43) Pub. Date: Nov. 10, 2022

(54) PROCESS FOR THE DI-O-ALKYLATION OF 1,3-DIOLS TO 1,3-DIETHERS

(71) Applicant: SABIC GLOBAL TECHNOLOGIES B.V., BERGEN OP ZOOM (NL)

(72) Inventors: Jaiprakash Brijlal SAINANI, Geleen (NL); Shirish Shrikant ABHYANKAR, Geleen (NL)

(21) Appl. No.: 17/623,449

(22) PCT Filed: Jun. 19, 2020
(86) PCT No.: PCT/EP2020/067167

§ 371 Date: Dec. 28, 2021

(30) Foreign Application Priority Data
Jul. 8, 2019 (EP) 19184860.5

(51) Int. Cl. C07C 41/14; C07C 41/40

(52) U.S. Cl. CPC C07C 41/14; C07C 41/40

(57) ABSTRACT

The present invention relates to a process for the di-O-alkylation of a 1,3-diol according to Formula I (I), said process comprising reacting said 1,3-diol with dioxane, an aliphatic or aromatic hydrocarbon solvent, an alkali metal hydroxide, and dimethyl sulphate, in order to obtain a 1,3-diether according to Formula II (II), wherein R¹ and R² are each independently a hydrogen atom or a hydrocarbyl group selected from alkyl, alkenyl, aryl, aralkyl, or alkylaryl groups, and one or more combinations thereof. The process according to the invention is an improved process for preparing 1,3-diether, such as 9,9-bis(methoxymethyl)fluorene, in a high yield and/or having a high purity. 9,9-bis(methoxymethyl)fluorene is a compound that is used as an electron donor for Ziegler-Natta catalysts.

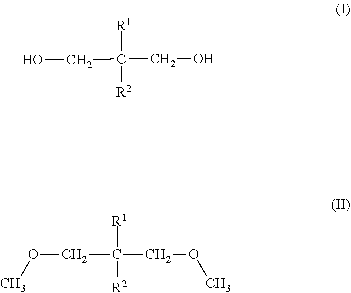

### BACKGROUND

The present invention relates to a novel process for the synthesis of 1,3-diethers, such as 9,9-bis(methoxymethyl)fluorene. The synthesis from fluorene via 9,9-bis(hydroxymethyl)fluorene to 9,9-bis(methoxymethyl)fluorene is known. 9,9-bis(methoxymethyl)fluorene is a compound that is used as an electron donor for Ziegler-Natta catalysts. The process for preparing this compound from fluorene typically comprises two separate steps, which are each discussed below.

Step 1 of the synthesis from fluorene to 9,9-bis(methoxymethyl)fluorene relates to the hydroxymethylation of fluorene to 9,9-bis(hydroxymethyl)fluorene. This hydroxymethylation is typically carried out using paraformaldehyde wherein dimethyl sulfoxide (DMSO) is used as a solvent. An alcoholic solution of sodium alkoxide, such as sodium methoxide in methanol or sodium ethoxide in ethanol is used as a base.

Typically, first a solution or dispersion of paraformaldehyde, DMSO and sodium alkoxide is prepared. Then a solution or dispersion of fluorene is added to this mixture. The reaction is usually carried out at very low temperatures in an ice bath. But temperatures of e.g. between 12 and 15° C. have also been disclosed in the prior art. The reaction is usually worked up by quenching with hydrochloric acid, addition of water and then one or more steps of extraction, distillation and/or recrystallization.

EP 0 728 770 discloses a method using a large volume of DMSO and a reaction temperature of 0° C. Chen et al. “synthesis of 9,9′-bis(methoxymethyl)fluorene”, Transactions of Tianjin University, 2003, volume 9, issue 3, pages 226-227 discloses that the reaction is carried out at a temperature of between 13 and 15° C. and requires a large volume of DMSO.

WO 2016/193212 A1 discloses a process for the synthesis of 9,9-bis(hydroxymethyl)fluorene from fluorene comprising providing a mixture of paraformaldehyde, dimethylsulfoxide and a sodium alkoxide and adding fluorene to said mixture to obtain 9,9-bis(hydroxymethyl)fluorene, wherein fluorene is added as a solid.

Step 2 of the synthesis from fluorene to 9,9-bis(methoxymethyl)fluorene relates to the etherification of 9,9-bis(hydroxymethyl)fluorene to 9,9-bis(methoxymethyl)fluorene. According to some prior art documents a combination of methyl iodide and sodium hydride is used, e.g. in EP 0 728 770. In Chen et al. a different process is disclosed using sodium hydroxide solution and dimethyl sulfate as the alkylating agent and a tetrabutylammonium bromide phase transfer agent. WO 2016/193212 A1 disclose a biphasic process for the synthesis of 9,9-bis(methoxymethyl)fluorine from 9,9-bis(hydroxymethyl)fluorine using an aqueous NaOH solution, tetrabutylammonium bromide as phase transfer catalyst, toluene as the organic phase and dimethyl sulfate as methylating agent.

JP H11 5760 describes the preparation of a 1,3-diether by reacting the corresponding 1,3-diol with sodium hydroxide and a halide, in particular alky halide, in dioxane as a solvent. However, any alky halide are relatively expensive compound.

The methods according to the prior art however have several disadvantages, such as non-optimal yield and impurity formation and expensive and non-commercially viable.

### SUMMARY

It is an object of the present invention to provide an improved process for preparing 1,3-diether, such as 9,9-bis(methoxymethyl)fluorene in a high yield.

It is another object of the present invention to provide an improved process for preparing 9,9-bis(methoxymethyl)fluorene having a high purity.

One or more of these aims are achieved by a process according to the present invention.

The present invention relates to a process for the di-O-alkylation of a 1,3-diol according to Formula I,

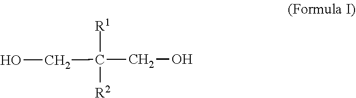

said process comprising reacting said 1,3-diol with dioxane, an aliphatic or aromatic hydrocarbon solvent, an alkali metal hydroxide, and dimethyl sulphate, in order to obtain a 1,3-diether according to Formula II,

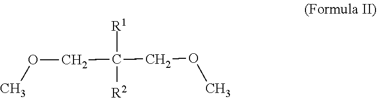

wherein R¹ and R² are each independently a hydrogen atom or a hydrocarbyl group selected from alkyl, alkenyl, aryl, aralkyl, or alkylaryl groups, and one or more combinations thereof.

With such process, which use dimethyl sulphate, in order to obtain a 1,3-diether according to Formula II allow to be commercially viable as dimethyl sulfate will be cheaper than any alkyl halide.

In addition, in the present invention, OH group is slightly acidic so it can extract proton with potassium hydroxide; whereas in JP H11 5760, OH group is not much acidic and hence NaH is required to abstract proton and form Na salt.

Furthermore, the use of NaH/methyl iodide will be costly not commercial viable.

Finally, if dioxane is replaced by DMF or DMSO or Acetonitrile as in the prior art, the reaction does not go to completion and end up in mixture which are difficult to purify.

### LIST OF DEFINITIONS

The following definitions are used in the present description and claims to define the stated subject matter. Other terms not cited below are meant to have the generally accepted meaning in the field.

“heteroatom” as used in the present description means: an atom other than carbon or hydrogen. However, as used herein—unless specified otherwise, such as below, —when “one or more hetereoatoms” is used one or more of the following is meant: F, CI, Br, I, N, O, P, B, S or Si.

“hydrocarbyl” as used in the present description means: is a substituent containing hydrogen and carbon atoms, or linear, branched or cyclic saturated or unsaturated aliphatic radical, such as alkyl, alkenyl, alkadienyl and alkynyl; alicyclic radical, such as cycloalkyl, cycloalkadienyl cycloalkenyl; aromatic radical, such as monocyclic or polycyclic aromatic radical, as well as combinations thereof, such as alkaryl and aralkyl.

“alkyl” as used in the present description means: an alkyl group being a functional group or side-chain consisting of carbon and hydrogen atoms having only single bonds. An alkyl group may be straight or branched and may be unsubstituted or substituted. It may or may not contain heteroatoms, such as oxygen (O), nitrogen (N), phosphorus (P), silicon (Si) or sulphur (S). An alkyl group also encloses aralkyl groups wherein one or more hydrogen atoms of the alkyl group have been replaced by aryl groups.

“aryl” as used in the present description means: an aryl group being a functional group or side-chain derived from an aromatic ring. An aryl group may be unsubstituted or substituted with straight or branched hydrocarbyl groups. It may or may not contain heteroatoms, such as oxygen (O), nitrogen (N), phosphorus (P), silicon (Si) or sulphur (S). An aryl group also encloses alkaryl groups wherein one or more hydrogen atoms on the aromatic ring have been replaced by alkyl groups.

“alkoxide” or “alkoxy” as used in the present description means: a functional group or side-chain obtained from a alkyl alcohol. It consist of an alkyl bonded to a negatively charged oxygen atom.

“Powder” as used in the present invention means: a solid substance composed of a large number of fine particles that may flow freely. It is in crystalline form. With fine particles is meant particles passing through a 50-100 mesh (viz. sieve of about 300 to about 150 micrometer). In other words, the particles have a diameter of between about 150 and about 300 micrometer, preferably at least 70%, more preferably 80%, more preferably 90%, even more preferably 95% of the particles have a diameter that lies in the range of between about 150 and about 300 micrometer.

“Granulate” as used in the present invention means: a solid substance composed of a large number of large particles that may flow freely. It is in crystalline form. With large particles is meant particles having a diameter of between about 300 micrometer and about 4.0 millimeter, preferably at least 70%, more preferably 80%, more preferably 90%, even more preferably 95% of the particles have a diameter that lies in the range of between about 300 micrometer and about 4.0 millimeter. Examples of granulates are pellets or flakes.

Unless stated otherwise, when it is stated that any R group is “independently selected from” this means that when several of the same R groups are present in a molecule they may have the same meaning of they may not have the same meaning. For example, for the compound R₂M, wherein R is independently selected from ethyl or methyl, both R groups may be ethyl, both R groups may be methyl or one R group may be ethyl and the other R group may be methyl.

### DESCRIPTION OF EMBODIMENTS

In an embodiment of the present invention, said hydrocarbyl group is be linear, branched or cyclic. Said hydrocarbyl group may be substituted or unsubstituted. Said hydrocarbyl group may contain one or more heteroatoms. Preferably, said hydrocarbyl group has from 1 to 10 carbon atoms, more preferably from 1 to 8 carbon atoms, even more preferably from 1 to 6 carbon atoms. Suitable examples of hydrocarbyl groups include alkyl-, cycloalkyl-, alkenyl-, alkadienyl-, cycloalkenyl-, cycloalkadienyl-, aryl-, aralkyl, alkylaryl, and alkynyl-groups.

In an embodiment of the present invention, the 1,3-diol is 9,9-bis(hydroxymethyl)fluorene, and the 1,3-diether is 9,9-bis(methoxymethyl)fluorene.

In an embodiment, the aliphatic or aromatic hydrocarbon solvent is a cyclic hydrocarbon. In an embodiment of this, the cyclic hydrocarbon is chosen from the group of toluene, benzene, cyclohexene and xylene. In a preferred embodiment, said solvent is toluene.

In an embodiment, the alkali metal hydroxide is in the form of a solid. In an embodiment, alkali metal hydroxide is in the form of a powder. Said alkali metal hydroxide in solid form can be in granulate form, for instance pellets or flakes, which can be ground to obtain a powder before addition.

In an embodiment, the reaction is carried out in a one phase system.

In an embodiment, as the alkali metal hydroxide, potassium hydroxide is used.

Potassium hydroxide is used to abstract proton more easily.

In case the reaction is diluted with water, the reaction will slow not or may not be completed.

However, with any alkyl halide, the reaction will not work with potassium hydroxide as it will hydrolyze.

In addition, dimethylsulphate decomposes slowly in alkali or alkaline solution.

However, potassium hydroxide with dimethylsulphate in presence of dioxane allow to obtain good reaction condition for dialkylation with high yield process is industrially viable and feasible.

In an embodiment of this, potassium hydroxide is used in an amount of between 4 and 10 moles per mole of mono- or dialcohol used.

In an embodiment, dimethyl sulfate is used in an amount of between 2.2 and 2.6 moles per mole of mono- or dialcohol used.

In an embodiment, the process comprises contacting a pre-mixture comprising the 1,3-diol, the dioxane, and a first portion of the hydrocarbon solvent with the alkali metal hydroxide, and subsequently adding dimethyl sulphate and a second portion of the hydrocarbon solvent. In embodiment of this, the hydrocarbon solvent is miscible with the pre-mixture. In another embodiment of this, the ratio of dioxane and hydrocarbon solvent in the pre-mixture is between 1:2 and 2:1, preferably between 1:1.5 and 1.5:1, more preferably between 1:1.1 and 1.1:1.

In an embodiment, the process comprises the steps of
i) providing a pre-mixture comprising the 1,3-diol, the dioxane, and a first portion of the hydrocarbon solvent;
ii) adding the hydroxide to the pre-mixture formed in step i) to form a mixture;
iii) adding the dimethyl sulfate and a second portion of the hydrocarbon solvent to the mixture formed in step ii) to form a reaction mixture;
iv) stirring the reaction mixture obtained in iii);
v) adding water to the reaction mixture obtained in step iv) and mixing;
vi) allowing the reaction mixture obtained in step v) to separate into an organic layer and a water layer;
vii) separate the organic layer formed in step vi) from the water layer;
viii) optionally washing the organic layer obtained in step vii) with water;
ix) optionally extracting the water layer obtained in step vii) with an additional portion of the hydrocarbon solvent to provide an organic extract layer;
x) evaporate the dioxane and the hydrocarbon solvent in the organic layer obtained in step viii) and optionally the organic extract layer obtained in step ix) to obtain crude 1,3-diether.

In an embodiment, mixing in step v) means stirring.

In an embodiment, in step iv) the reaction mixture is stirred until completion. With “until completion” in this context is meant that at most 10 wt. % of the starting material has remained in the reaction mixture, preferably at most 5 wt. %, more preferably at most 1 wt. %.

In an embodiment, step viii) entails the following: adding water to the organic layer obtained in step vii) and mixing; allowing the mixture to separate into an organic layer and a water layer; and separating said organic layer from the water layer.

In an embodiment, the crude 1,3-diether obtained in step x) is recrystallized to obtain 1,3-diether, preferably using an alcohol solvent, more preferably methanol. Recrystallization can for instance be performed with alcohol such as ethanol, isopropanol or methanol. Preferably, recrystallization is performed with methanol. In an embodiment, between 1000 and 1200 milliliter of methanol per mole of 9,9-bis(hydroxymethyl)fluorene used for the recrystallization.

In an embodiment, the stirring in step iv) is carried out for a period of between 6 and 8 hours.

In an embodiment, step iii) and/or step iv) are carried out at a temperature of between 15 and 20° C.

In an embodiment, in step i) and dioxane is used in an amount of between 400 and 500 milliliter per mole of the 1,3-diol used. In an embodiment, in step i) the hydrocarbon solvent is toluene, used in an amount of between 400 and 500 milliliter per mole the 1,3-diol used.

In an embodiment, in step ii) the hydroxide is solid potassium hydroxide, and this is added in an amount of between 4 and 10 mole per mole of the 1,3-diol used.

In an embodiment, in step iii) dimethyl sulfate is added in an amount of between 2.2 to 2.6 mole dimethyl sulfate per mole of the 1,3-diol. In an embodiment, the solvent is toluene, of which between 400 and 500 milliliter toluene per mole of the 1,3-diol used. In a specific embodiment, a mixture of dimethyl sulfate and toluene is added gradually, preferably dropwise, to the reaction mixture of step ii) over a period of between 2 and 3 hours at a temperature of between 15 and 20° C.

In an embodiment, in step iv) the reaction mixture is stirred for a period of between 6 and 8 hours at a temperature of between 15 and 20° C.

In an embodiment, in step v) water is added in an amount of between 600 and 800 milliliter per mole of the 1,3-diol used and stirred for a period of time between 5 and 25 minutes at a temperature of between 15 and 20° C.

In an embodiment, in step viii) the organic layer is washed with water in an amount of between 400 and 500 milliliter water per mole of the 1,3-diol used;

In an embodiment, in step ix) the hydrocarbon solvent is toluene, which is used in an amount of between 100 and 250 milliliter per mole of the 1,3-diol used.

In an embodiment of the present invention, the process comprises the steps of
i) providing a pre-mixture of 9,9-bis(hydroxymethyl)fluorene, dioxane in an amount of between 400 and 500 milliliter per mole of 9,9-bis(hydroxymethyl)fluorene used, and toluene in an amount of between 400 and 500 milliliter per mole 9,9-bis(hydroxymethyl)fluorene used;
ii) adding to the pre-mixture of step i) solid potassium hydroxide in an amount of between 4 and 10 mole per mole of 9,9-bis(hydroxymethyl)fluorene used to obtain a reaction mixture;
iii) adding gradually, preferably dropwise, to the reaction mixture of step ii) a mixture of dimethyl sulfate in an amount of between 2.2 to 2.6 mole dimethyl sulfate per mole of 9,9-bis(hydroxymethyl)fluorene and toluene and between 400 and 500 milliliter toluene per mole of 9,9-bis(hydroxymethyl)fluorene used over a period of between 2 and 3 hours at a temperature of between 15 and 20° C.;
iv) stirring the reaction mixture obtained in step iii) for a period of between 6 and 8 hours at a temperature of between 15 and 20° C.;
v) adding water to the reaction mixture obtained in step iv) in an amount of between 600 and 800 milliliter per mole of 9,9-bis(hydroxymethyl)fluorene used and stirring for a period of time between 5 and 25 minutes at a temperature of between 15 and 20° C.;
vi) allowing the reaction mixture obtained in step v) to separate in an organic layer and a water layer;
vii) separate the organic layer formed in step iv) from the water layer;
viii) washing the organic layer obtained in step vii) with water in an amount of between 400 and 500 milliliter water per mole of 9,9-bis(hydroxymethyl)fluorene used;
ix) extracting the water layer obtained in step vii) with toluene in an amount of between 100 and 250 milliliter per mole of 9,9-bis(hydroxymethyl)fluorene used to obtain an organic extract layer;
x) evaporating the dioxane and the toluene from the organic layer formed in step vii) and the organic extract layer obtained in step ix) to obtain crude 9,9-bis(methoxymethyl)fluorene;
xii) recrystallize the crude 9,9-bis(methoxymethyl)fluorene from between 1000 and 1200 milliliter of methanol per mole of 9,9-bis(hydroxymethyl)fluorene used and dry under vacuum to obtain 9,9-bis(methoxymethyl)fluorene.

Examples of 1,3-diethers that can be prepared using the present invention according to Formula II include 1,3-dimethoxypropane, 1,3-diethoxypropane, 1-methoxy-3-ethoxypropane, 1-methoxy-3-butoxypropane, 1-methoxy-3-cyclohexoxypropane, 2,2-dimethyl-1,3-dimethoxypropane, 2,2-diethyl-1,3-dimethoxypropane, 2,2-di-n-butyl-1,3-dimethoxypropane, 2,2-diiso-butyl-1,3-dimethoxypropane, 2-ethyl-2-n-butyl-1,3-dimethoxypropane, 2-n-propyl-2-cyclopentyl-1,3-dimethoxypropane, 2,2-dimethyl-1,3-diethoxypropane, 2-n-propyl-2-cyclohexyl-1,3-diethoxypropane, 2-(2-ethylhexyl)-1,3-dimethoxypropane, 2-isopropyl-1,3-dimethoxypropane, 2-n-butyl-1,3-dimethoxypropane, 2-sec-butyl-1,3-dimethoxypropane, 2-cyclohexyl-1,3-dimethoxypropane, 2-phenyl-1,3-diethoxypropane, 2-cumyl-1,3-diethoxypropane, 2-(2-phenyllethyl)-1,3-dimethoxypropane, 2-(2-cyclohexylethyl)-1,3-dimethoxypropane, 2-(p-chlorophenyl)-1,3-dimethoxypropane, 2-(diphenylmethyl)-1,3-dimethoxypropane, 2-(1-naphthyl)-1,3-dimethoxypropane, 2-(fluorophenyl)-1,3-dimethoxypropane, 2-(1-decahydronaphthyl)-1,3-dimethoxypropane, 2-(p-t-butylphenyl)-1,3-dimethoxypropane, 2,2-dicyclohexyl-1,3-dimethoxypropane, 2,2-di-n-propyl-1,3-dimethoxypropane, 2-methyl-2-n-propyl-1,3-dimethoxypropane, 2-methyl-2-benzyl-1,3-dimethoxypropane, 2-methyl-2-ethyl-1,3-dimethoxypropane, 2-methyl-2-phenyl-1,3-dimethoxypropane, 2-methyl-2-cyclohexyl-1,3-dimethoxypropane, 2,2-bis(pchlorophenyl)-1,3-dimethoxypropane, 2,2-bis(2-cyclohexylethyl)-1,3 dimethoxypropane, 2-methyl-2-iso butyl-1,3-dimethoxypropane, 2-methyl-2-(2-ethylhexyl)-1,3-dimethoxy propane, 2-methyl-2-isopropyl-1,3-dimethoxypropane, 2,2-diphenyl-1,3-dimethoxypropane, 2,2-dibenzyl-1,3-dimethoxypropane, 2,2-bis(cyclohexylmethyl)-1,3-dimethoxypropane, 2,2-diiso butyl-1,3-diethoxypropane, 2-iso butyl-2-isopropyl-1,3-dimethoxypropane, 2,2-di-sec-butyl-1,3-dimethoxypropane, 2,2-di-t-butyl-1,3-dimethoxypropane, 2,2-dineopentyl-1,3-dimethoxypropane, 2-isopropyl-2-isopentyl-1,3-dimethoxypropane, 2-phenyl-2-benzyl-1,3-dimethoxypropane, 2-cyclohexyl-2-cyclohexylmethyl-1,3-dimethoxypropane, 2-isopropyl-2-(3, 7-dimethyloctyl) 1,3-dimethoxypropane, 2,2-diisopropyl-1,3-dimethoxypropane, 2-isopropyl-2-cyclohexylmethyl-1,3-dimethoxypropane, 2,2-diisopentyl-1,3-dimethoxypropane, 2-isopropyl-2-cyclohexyl-1,3-dimethoxypropane, 2-isopropyl-2-cyclopentyl-1,3-dimethoxypropane, 2,2-dicylopentyl-1,3-dimethoxypropane, 2-n-heptyl-2-n-pentyl-1,3-dimethoxypropane, 9,9-bis(methoxymethyl)fluorene, 1,3-dicyclohexyl-2,2-bis(methoxymethyl)propane, 3,3-bis(methoxymethyl)-2,5-dimethylhexane, or any combination of the foregoing. In an embodiment, the internal electron donor is 1,3-dicyclohexyl-2,2-bis(methoxymethyl)propane, 3,3-bis(methoxymethyl)-2,5-dimethylhexane, 2,2-dicyclopentyl-1,3-dimethoxypropane and combinations thereof.

A specific example of a compound according to Formula II is 9,9-bis (methoxymethyl) fluorene:

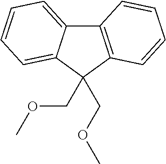 [SMILES: COCC1(COC)c2ccccc2-c2ccccc21]

In an embodiment, the 1,3-diol compound according to Formula I is 9,9-bis(methoxymethyl)fluorene.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage.

The scope of the present invention is defined by the appended claims. One or more of the objects of the invention are achieved by the appended claims.

### EXAMPLES

The present invention is further elucidated based on the Examples below which are illustrative only and not considered limiting to the present invention.

### Example 1—Procedure for Preparation of 9,9-bis(methoxymethyl)fluorine (BMMF)

To a two litre round bottom flask is added 200 mL dioxane, 200 mL toluene and 100 g (0.44 moles) 9,9-bis(hydroxymethyl)fluorine (BHMF) under stirring. The mixture is cooled to room temperature. To this mixture, 173.7 g (3.1 moles) potassium hydroxide is added. The mixture is stirred for 30 minutes. Gradually 133.8 g (1.06 moles) dimethylsulphate (DMS) and 200 mL of toluene is added over the course of 2-3 hours at a temperature of 15-20° C. The reaction mixture is subsequently stirred at the same temperature until the reaction is complete. The reaction progress is checked using Thin Layer Chromatography (TLC) with a diluted sample of approximately 2-5%. For the TLC a mixture of 20% ethylacetate and 80% heptane is used. The reaction is considered complete when 9,9-bis(hydroxymethyl)fluorine is absent from the TLC plate, and the 9,9-bis(methoxymethyl)fluorine is present in majority quantity, possible with a faint spot of mono-O-alkylated 9,9-bis(hydroxymethyl)fluorine. The reaction progress can also be monitored with gas chromatography (GC). The reaction is then considered complete when more than 95% is 9,9-bis(methoxymethyl)fluorine. If the reaction is not complete, stirring is continued for 1-2 hours at room temperature before the reaction progress is checked again.

After completion of the reaction, 300 mL of water is added. The mixture is stirred for 15 minutes and the organic layer is separated. If the organic layer is not clear, it can be filtered through a Hyflow bed. After separation, the organic layer is washed with 200 mL water, and the aqueous layer is extracted with 50-100 mL toluene. The combined toluene and dioxane layer is distilled and the last traces are removed by applying vacuum. A solid residue (85-90 g) is obtained. To this solid residue, 500 mL methanol is added, and the mixture is refluxed for 15-30 minutes. The mixture is subsequently cooled to 10° C. and maintained at that temperature for 2 hours. The mixture is filtered, and the solid product is washed twice with 25 mL of chilled methanol. The product is dried at 50-60° C. under vacuum.

Purity of the product is determined by Gas Chromatography. If the purity is less then desired, 100-150 methanol can be added to make a slurry of the product, and the product can be filtered. Distilling the methanol allows to recover the 9,9-bis(methoxymethyl)fluorine.

The wet weight of the product was 85 gram. The dry weight of 9,9-bis(methoxymethyl)fluorine obtained was 70-80 gram. The crude product obtained had a purity of >95% as assessed with Gas Chromatography. Finally purified using methanol gave more than 90% yield and purity of more than 99% as assessed by Gas Chromatography. The appearance of the product is white to off-white.

### Example 2

Charge 10 mL dioxane, 10 mL toluene and 5 g (0.022 moles) 9,9-bis(hydroxymethyl)fluorine (BHMF) under stirring. To this mixture, 6.2 g (0.155 moles) sodium hydroxide added.

The mixture is stirred for 30 minutes. Gradually 7.85 g (0.055 moles) iodomethane is added over the course of 30 min at a temperature of 15-20° C.

The reaction mixture is subsequently stirred at the same temperature and progress is checked using Thin Layer Chromatography (TLC) with a diluted sample of approximately 2-5%.

For the TLC a mixture of 20% ethyl acetate and 80% heptane is used.

The reaction was not complete after 24 hours stirring and shows about 20% unreacted 9,9-bis(hydroxymethyl)fluorine, 9,9-bis(methoxymethyl)fluorine about 50% as major and mono-O-alkylated 9,9-bis(hydroxymethyl)fluorine.

The reaction was checked again but there was no further progress.

Substituting iodomethane for dimethyl sulfate, dialkylation does not go to completion.

### Example 3

Charge 10 mL dioxane, 10 mL toluene and 5 g (0.022 moles) 9,9-bis(hydroxymethyl)fluorine (BHMF) under stirring. To this mixture, 6.2 g (0.155 moles) sodium hydroxide and water (20 ml) is added.

The mixture is stirred for 30 minutes. Gradually 7.85 g (0.055 moles) iodomethane is added over the course of 30 min at a temperature of 15-20° C.

The reaction mixture is subsequently stirred at the same temperature and progress is checked using Thin Layer Chromatography (TLC) with a diluted sample of approximately 2-5%.

For the TLC a mixture of 20% ethyl acetate and 80% heptane is used.

The reaction was slow and was not complete after 24 hours stirring and shows about 65% unreacted 9,9-bis(hydroxymethyl)fluorine, 9,9-bis(methoxymethyl)fluorine about 30% and mono-O-alkylated 9,9-bis(hydroxymethyl)fluorine.

The reaction was checked again but there was no further progress.

Using 30% caustic solution it is found reaction is very slow and does not go to completion after overnight stirring.

1. A process for the di-O-alkylation of a 1,3-diol according to Formula I, 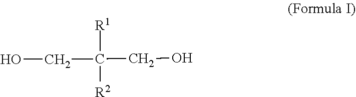
said process comprising reacting said 1,3-diol with dioxane, an aliphatic or aromatic hydrocarbon solvent, an alkali metal hydroxide, and dimethyl sulphate, in order to obtain a 1,3-diether according to Formula II, 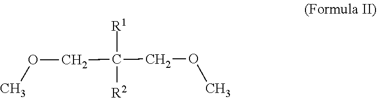
wherein R¹ and R² are each independently a hydrogen atom or a hydrocarbyl group selected from an alkyl group, an alkenyl group, an aryl group, and aralkyl group, an alkylaryl groups, or one or more combinations thereof.

2. Process according to claim 1, wherein the 1,3-diol is 9,9-bis(hydroxymethyl)fluorene, and the 1,3-diether is 9,9-bis(methoxymethyl)fluorene.

3. Process according to claim 1, wherein the aliphatic or aromatic hydrocarbon solvent is a cyclic hydrocarbon.

4. Process according to claim 1, wherein the alkali metal hydroxide is in the form of a solid.

5. Process according to claim 1, wherein the reaction is carried out in a one phase system.

6. Process according to claim 1, wherein the alkali metal hydroxide is potassium hydroxide.

7. Process according to claim 1, wherein dimethyl sulfate is used in an amount of between 2.2 and 2.6 moles per mole of mono- or dialcohol used.

8. Process according to claim 1, comprising contacting a pre-mixture comprising the 1,3-diol, the dioxane, and a first portion of the hydrocarbon solvent with the alkali metal hydroxide, and subsequently adding dimethyl sulphate and a second portion of the hydrocarbon solvent.

9. Process according to claim 8, wherein the hydrocarbon solvent is miscible with the pre-mixture.

10. Process according to claim 8, wherein the ratio of dioxane and hydrocarbon solvent in the pre-mixture is between 1:2 and 2:1.

11. Process according to claim 1, comprising the steps of
i) providing a pre-mixture comprising the 1,3-diol, the dioxane, and a first portion of the hydrocarbon solvent;
ii) adding the hydroxide to the pre-mixture formed in step i) to form a mixture;
iii) adding the dimethyl sulfate and a second portion of the hydrocarbon solvent to the mixture formed in step ii) to form a reaction mixture;
iv) stirring the reaction mixture obtained in iii);
v) adding water to the reaction mixture obtained in step iv) and mixing;
vi) allowing the reaction mixture obtained in step v) to separate into an organic layer and a water layer;
vii) separate the organic layer formed in step vi) from the water layer;
viii) optionally washing the organic layer obtained in step vii) with water;
ix) optionally extracting the water layer obtained in step vii) with an additional portion of the hydrocarbon solvent to provide an organic extract layer;
x) evaporate the dioxane and the hydrocarbon solvent in the organic layer obtained in step viii) and optionally the organic extract layer obtained in step ix) to obtain crude 1,3-diether.

12. Process according to claim 11, wherein the crude 1,3-diether obtained in step x) is recrystallized to obtain 1,3-diether.

13. Process according to claim 11, wherein the stirring in step iv) is carried out for a period of between 6 and 8 hours.

14. Process according to claim 11, wherein step iii) and/or step iv) are carried out at a temperature of between 15 and 20° C.

15. Process according to claim 1, comprising the steps of
i) providing a pre-mixture of 9,9-bis(hydroxymethyl)fluorene, dioxane in an amount of between 400 and 500 milliliter per mole of 9,9-bis(hydroxymethyl)fluorene used, and toluene in an amount of between 400 and 500 milliliter per mole of 9,9-bis(hydroxymethyl)fluorene used;
ii) adding to the pre-mixture of step i) solid potassium hydroxide in an amount of between 4 and 10 mole per mole of 9,9-bis(hydroxymethyl)fluorene used to obtain a reaction mixture;
iii) adding gradually, to the reaction mixture of step ii) a mixture of dimethyl sulfate in an amount of between 2.2 to 2.6 mole dimethyl sulfate per mole of 9,9-bis(hydroxymethyl)fluorene and toluene and between 400 and 500 milliliter toluene per mole of 9,9-bis(hydroxymethyl)fluorene used over a period of between 2 and 3 hours at a temperature of between 15 and 20° C.;
iv) stirring the reaction mixture obtained in step iii) for a period of between 6 and 8 hours at a temperature of between 15 and 20° C.;
v) adding water to the reaction mixture obtained in step iv) in an amount of between 600 and 800 milliliter per mole of 9,9-bis(hydroxymethyl)fluorene used and stirring for a period of time between 5 and 25 minutes at a temperature of between 15 and 20° C.;
vi) allowing the reaction mixture obtained in step v) to separate in an organic layer and a water layer;
vii) separate the organic layer formed in step iv) from the water layer;
viii) washing the organic layer obtained in step vii) with water in an amount of between 400 and 500 milliliter water per mole of 9,9-bis(hydroxymethyl)fluorene used;
ix) extracting the water layer obtained in step vii) with toluene in an amount of between 100 and 250 milliliter per mole of 9,9-bis(hydroxymethyl)fluorene used to obtain an organic extract layer;
x) evaporating the dioxane and the toluene from the organic layer formed in step vii) and the organic extract layer obtained in step ix) to obtain crude 9,9-bis(methoxymethyl)fluorene; and
xii) recrystallize the crude 9,9-bis(methoxymethyl)fluorene from between 1000 and 1200 milliliter of methanol per mole of 9,9-bis(hydroxymethyl)fluorene used and drying under vacuum to obtain 9,9-bis(methoxymethyl)fluorene.
